SEQUENCE LISTING (2 sequences)

SEQ ID NO: 1 - DNA, 38 bp, artificial sequence
  primer sequence used to amplify mStrawberry gene
gatctcgagcgtgagcaagggcgaggagaataacatgg

SEQ ID NO: 2 - DNA, 38 bp, artificial sequence
  primer sequence used to amplify mStrawberry gene
tcagcggccgctacttgtacagctcgtccatgccgccg

(19) United States
(12) Patent Application Publication
(10) Pub. No.: US 2009/0214663 A1
(43) Pub. Date: Aug. 27, 2009

(54) Virus coated nanoparticles and uses thereof

(76) Inventors: Thomas B. Albrecht, Galveston, TX (US); Robert A. Davey, Galveston, TX (US)

Correspondence Address: Benjamin Aaron Adler;ADLER & ASSOCIATES, 8011 Candle Lane, Houston, TX, 77071, US

(21) Appl. No.: 12/383,744

(22) Filed: Mar. 26, 2009

Related U.S. Application Data
(63) Continuation-in-part of application No. PCT/US2007/020723, filed on Sep. 26, 2007.
(60) Provisional application No. 60/847,219, filed on Sep. 26, 2006.

(51) Int. Cl. A61K 9/14; B32B 5/16; B29C 47/00; A61K 38/02; A61K 39/00; A61K 39/12; C12Q 1/70

(52) U.S. Cl. 424/491; 428/403; 264/7; 514/2; 424/184.1; 424/204.1; 435/5

(57) ABSTRACT

The present invention discloses method to coat nanoparticles with viral envelope containing specific proteins. The present invention also discloses that such viral envelope coated nanoparticles can be targeted to specific cells and cellular entry pathway, thereby permitting their use as vaccines, in targeted delivery of therapeutic products and in the study of virus adsorption, cell penetration and viral entry pathways.

### CROSS-REFERENCE TO RELATED APPLICATIONS

This is a continuation-in-part under 35 U.S.C. §120 of International Application PCT/US2007/020723, with an international filing date of Sep. 26, 2007, now abandoned, which under 35 U.S.C. §119(e) claims priority to provisional application U.S. Ser. No. 60/847,219, filed Sep. 26, 2006, now abandoned.

### FEDERAL FUNDING LEGEND

This invention was produced in part using funds obtained through National Institute of Health grant numbers ES10018 and DE11389. Consequently, the federal government has certain rights in this invention.

### BACKGROUND OF THE INVENTION

1. Field of the Invention

The present invention relates to the field of nanotechnology and virology. More specifically, the present invention discloses a method of coating nanoparticles (NPs) with virus envelopes containing specific proteins that facilitate the targeting of specific cells and cellular entry pathways and the use of such particles as vaccines, in the targeted delivery of therapeutic products, study of virus adsorption, cell penetration and virus entry pathways.

2. Description of the Related Art

In the field of nanotechnology, the bulk of work has been devoted to the assembly of nanoparticles that encapsulate drugs effectively, have low immunogenicity and avoid being removed from circulation. Many formulations exist that are based on lipids, carbohydrates, polymers and proteins, and many of these have been tested in animal models. Nanoparticles with the longest circulatory half-life should have hydrophilic coats and are about 100 nm in size. These two parameters describe most viruses. Most have a hydrophilic protein+carbohydrate shell that encapsulate a core of between 30 to 200 nm in diameter. The capsid core contains the viral RNA or DNA genome, a cargo that is efficiently delivered to the cytoplasm of the cell where it replicates (or is trafficked to the nucleus). Indeed, virus capsid proteins have been used to construct nanoparticles as a gene delivery vehicle. However, these were used to stabilize DNA for cells to adsorb, more than a method to target genes to specific cell types.

An important problem is how to target nanoparticles to specific tissues, organs, tumors or cell types. This problem has been addressed previously by using antibody or peptide-based ligands that bind to cell surface molecules. While certain types of tumor cells have been successfully bound by ligand-modified nanoparticles, efficient penetration into the cell cytoplasm has not been achieved. These ligands were essentially static in nature and most nanoparticles end being held to the cell surface. Another outcome was inefficient endocytosis, after which the nanoparticle ends up in lysosomes, a low pH environment rich in proteases, that destroy many therapeutic agents.

Thus, prior art is deficient in a method that would enable the efficient delivery of nanoparticle cargoes to specific cells, specific subcellular organelles and into the cytoplasm. The present invention fulfills this long-standing need and desire in the art.

### SUMMARY OF THE INVENTION

In one embodiment of the present invention, there is provided a composition comprising a biodegradable core particle having a diameter of at least 100 nm and partial hydrophobic properties on unmodified surface of the core particle and a coating comprising one or more than one viral envelope proteins.

In another related embodiment of the present invention, there is provided a method of generating the viral envelope coated core particle discussed supra. This method comprises lysing an intact virus via an osmotic shock and sonicating membrane of the virus to dissociate viral envelope and nucleocapsid of the virus. The viral envelope and the nucleocapsid of the virus is then separated using a density gradient. This is followed by incubation of the viral envelope and the core particle for at least fifteen minutes. The viral envelope/core particle mixture is then sonicated to dissociate envelope vesicle aggregates and to permit association of the envelope with the core particle. Subsequently, the virus envelope/core particle mixture is passed through an extruder with a defined pore size from 50 to about 200 nm such that the passage through the filter and pressure applied during the passage forces the membrane of the virus to be extruded over the core particle, thereby generating the viral envelope coated core particle.

In yet another related embodiment of the present invention, there is provided a method of targeted therapy to an individual. This method comprises administering the above-discussed composition to the individual, where the viral envelope protein in the composition targets the composition to specific receptors on a cell, to specific cellular entry mechanisms within the targeted cell or to a combination thereof.

In still yet another related embodiment of the present invention, there is provided an immunogenic composition. Such a composition comprises a nucleic acid or a nucleic acid like molecule encoding an immunogenic peptide or an antigen, an immunogenic peptide, a protein or an immune stimulant.

In another related embodiment of the present invention, there is provided a method of delivering an immunogenic composition to an immune cell in an individual. This method comprises administering the above-discussed composition to the individual, where the viral envelope protein in the composition binds specifically to the immune cell, thereby delivering the immunogenic composition to the immune cell in the individual.

In yet another related embodiment of the present invention there is provided a kit. Such a kit comprises the above-discussed composition, where the composition comprises a protein of a pathogen or a modified protein of a pathogen.

In still yet another related embodiment of the present invention, there is provided a method of detecting an infection caused by a pathogen in an individual. Such a method comprises obtaining a biological sample from the individual and contacting the biological sample with the kit discussed supra, thereby detecting the infection caused by the pathogen in the individual.

### BRIEF DESCRIPTION OF THE DRAWINGS

FIGS. 1A-1H show the steps involved in coating of nanoparticles with Moloney murine leukemia virus (Mo-MLV) envelope containing membranes. FIG. 1A shows disruption of the virus by osmotic shock followed by sonication to further separate envelope (env)-containing membranes from other virus components including the nucleocapsid (core) (FIG. 1B). In FIG. 1C, intact virus and cores were pelleted by (1) centrifugation at 20,000 g, and then (2) virus membranes present in the supernatant were pelleted at 100,000 g. FIG. 1D shows incubation of purified virus membranes with nanoparticles, followed by sonication to disrupt large membranes-NP aggregates (FIG. 1E). Membranes were forced over nanoparticles by extrusion of the NP-membrane mixture through a 0.2 μM polycarbonate filter using a mini-extruder (Avanti Polar Lipids, Inc.) (FIG. 1F). In FIG. 1G, Mo-MLV membrane associated nanoparticles (Mo-NP) were separated from uncoated nanoparticles and residual membranes on a 0-27% (w/v) dextran gradient and used for assays with cells (FIG. 1H).

FIGS. 2A-2B show that extrusion efficiently coats nanoparticles with lipid membranes. Rhodamine (red fluorescent)-labeled phosphotidylethanolamine was mixed with brain lipids (Avanti polar lipids) at a 1:99 ratio (w/w) and dried. In FIG. 2A, the dried lipid was resuspended in PBS, extruded over green fluorescent nanoparticles of 100 nm in diameter, and separated on a 0-27% (w/v) dextran (70 kDa) gradient (right pane). Intact nanoparticles (left panel) and lipids alone (middle panel) were also applied to the gradients. Lipids remained at the surface of the gradient (red arrowhead) while nanoparticles migrated midway down the gradient (green arrowhead). Images of the tubes containing the gradients were taken using long wavelength UV illumination. A band of low-density fluorescent material was also visible at the surface of the gradient. This was a contaminant in the dextran that separated away from the nanoparticles.

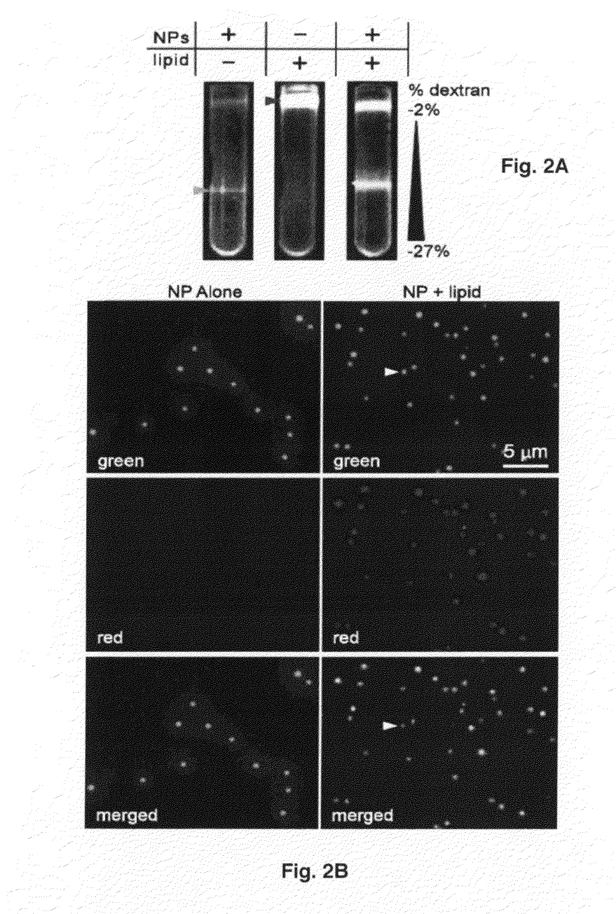

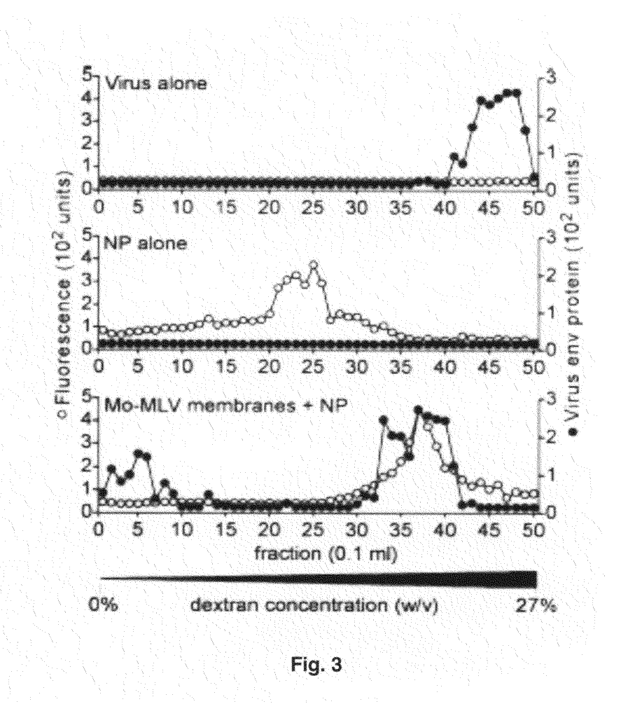

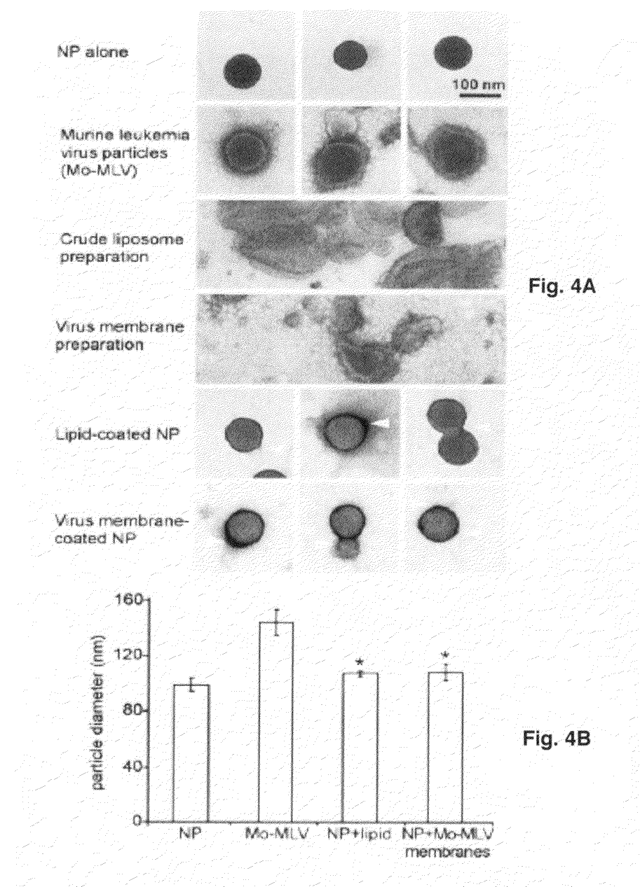

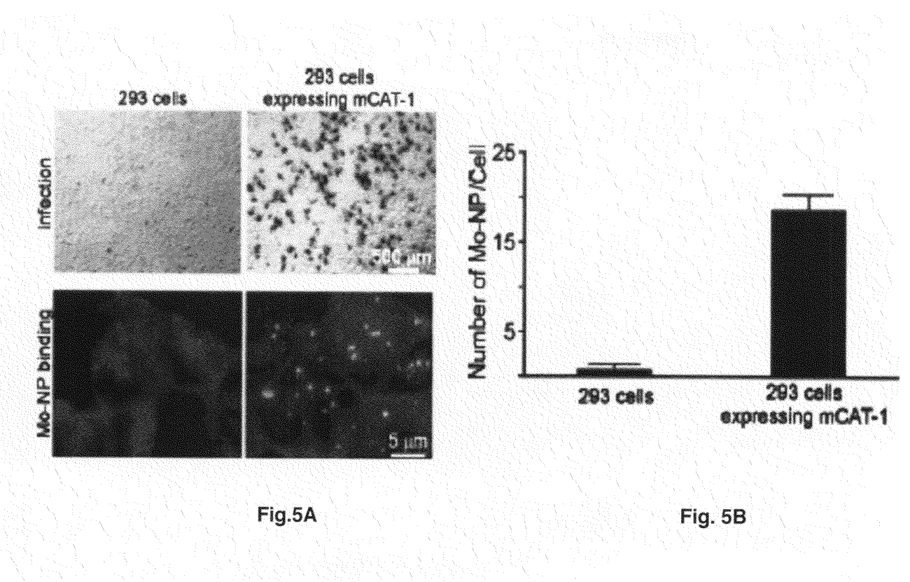

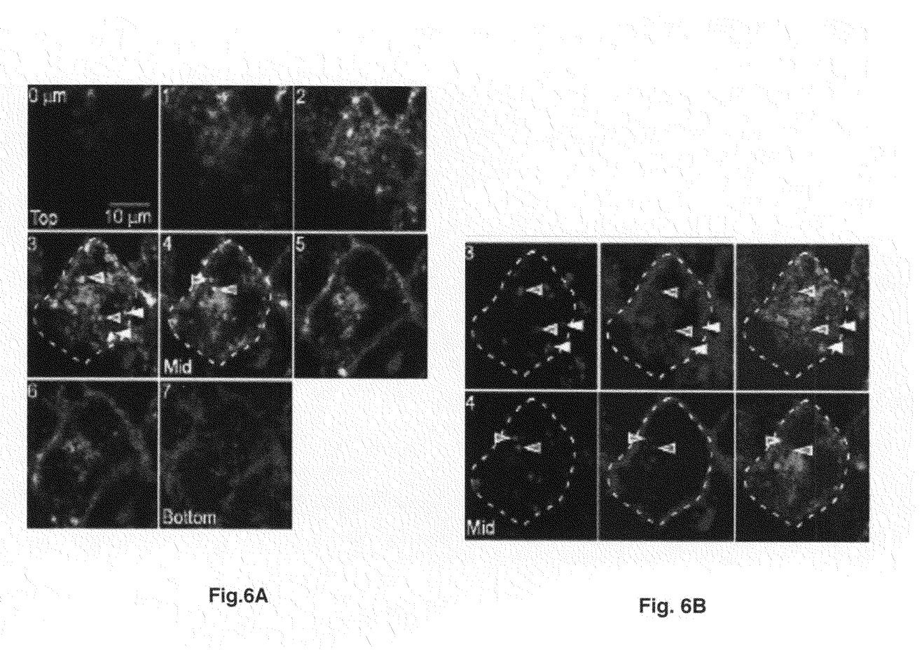

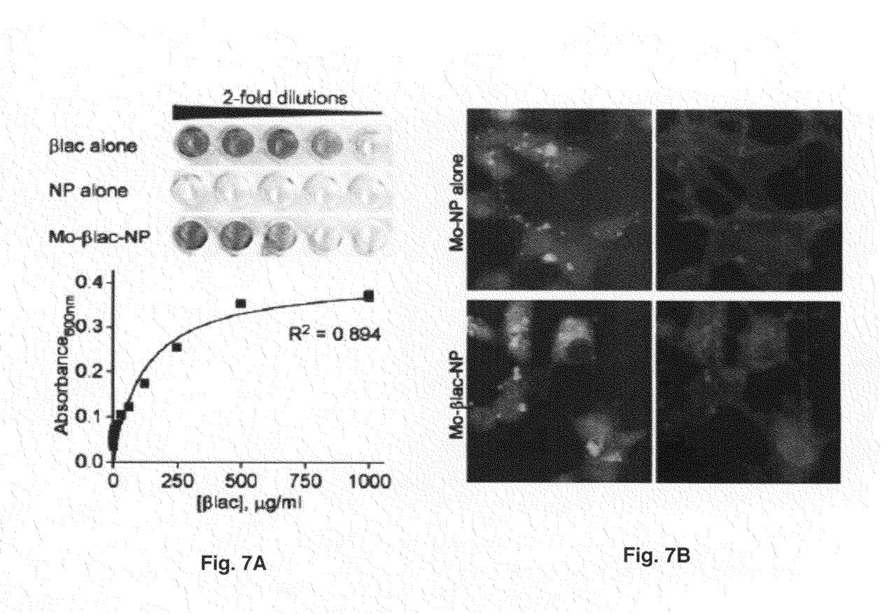

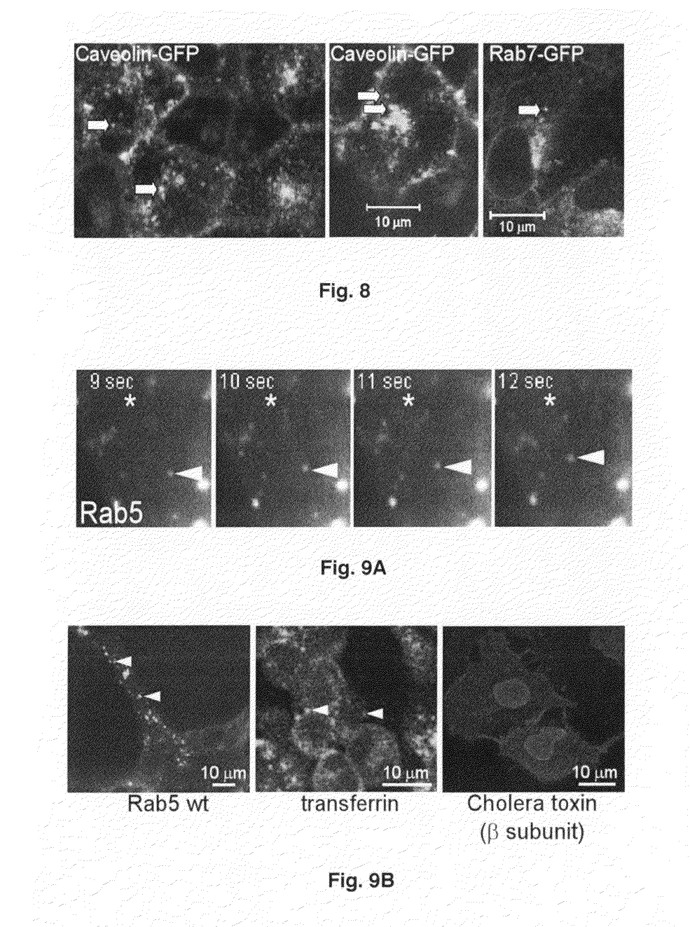

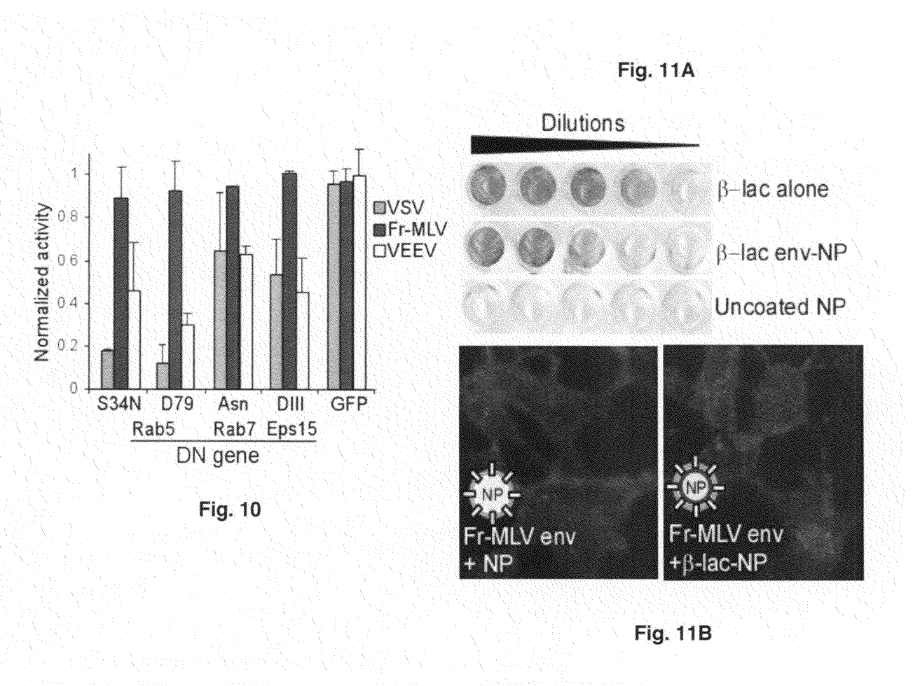

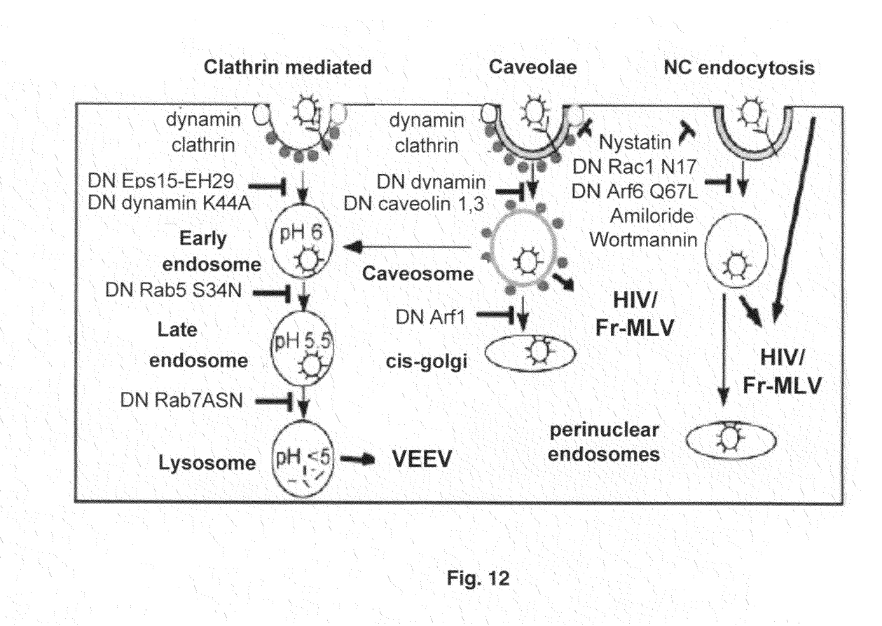

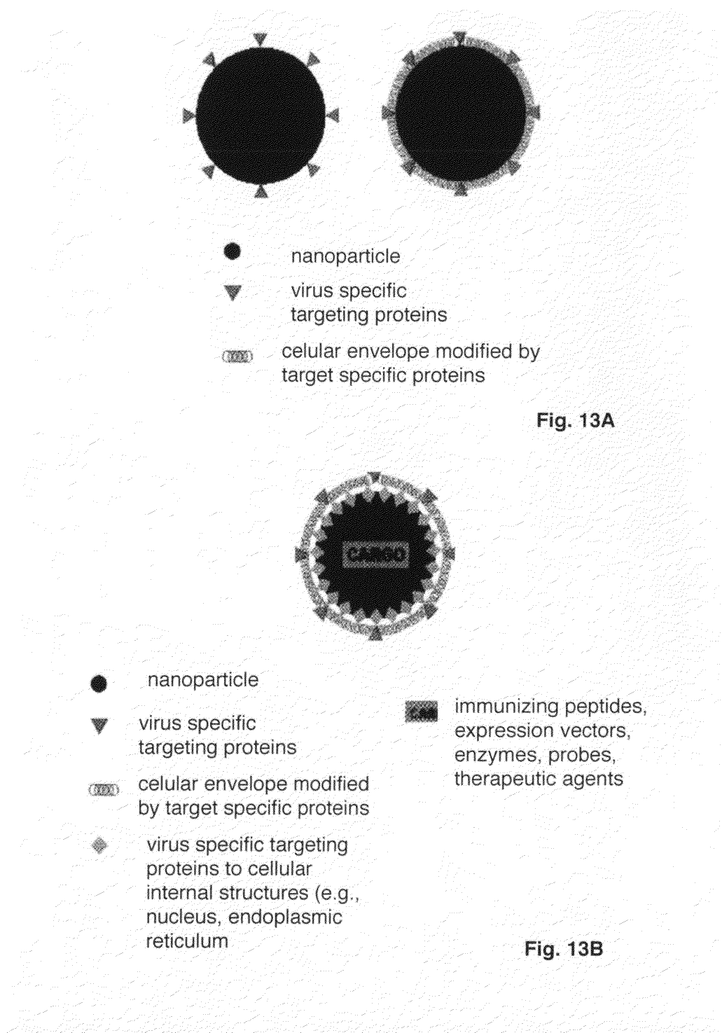

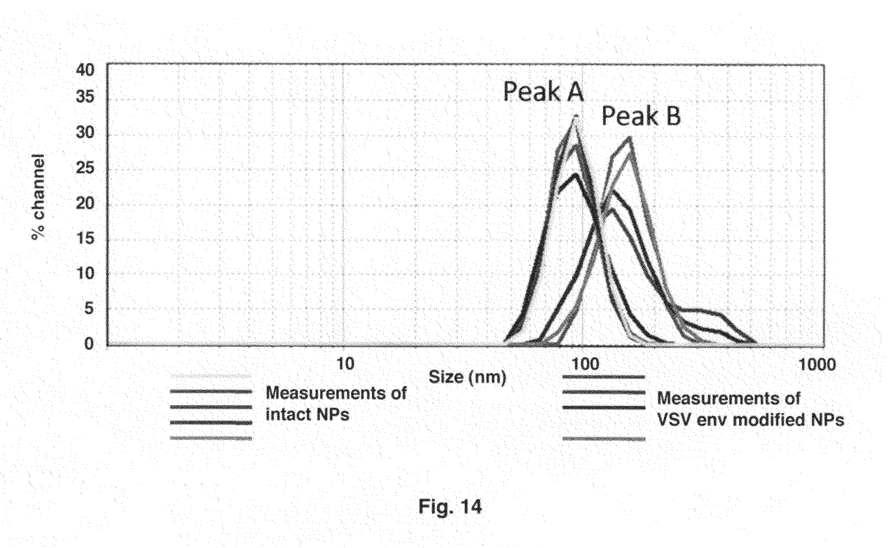

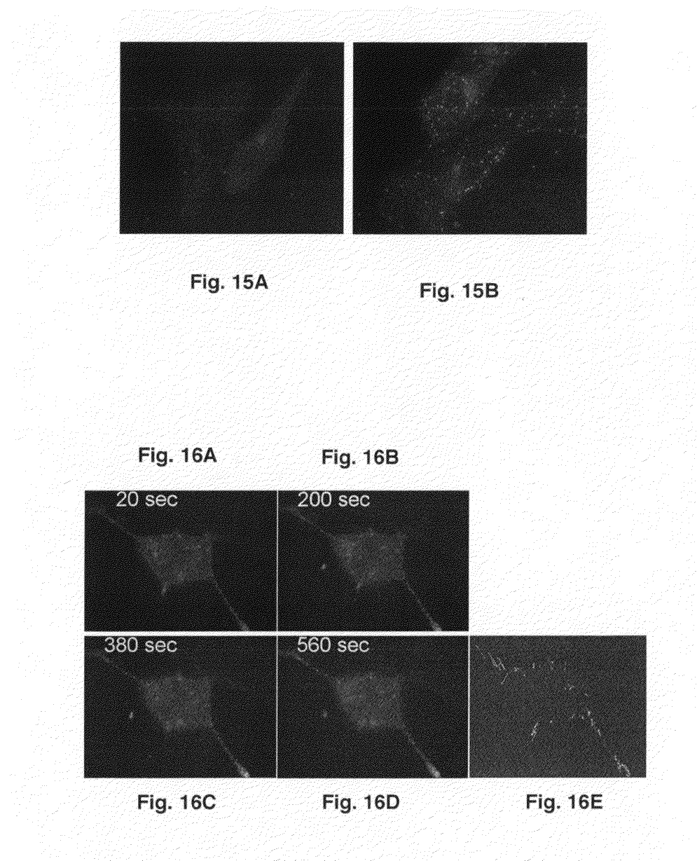

In FIG. 2B, fractions corresponding to peak fluorescence were collected from the NP alone (left panels) or NP+lipid (right panels) gradients and analyzed for green and red fluorescence by microscopy. The samples were diluted (approximately 1:1000) and smears were made on microscope slides such that the nanoparticles appeared as distinct points of light. The composite of green and red fluorescence images (merged) shows the efficiency of NP coating by lipid, where coated particles appear orange. A single NP that was not coated is indicated by white arrowheads. Images were taken using a 100× oil immersion objective lens. The NPs behave as point light sources with some flaring of the emitted light making each particle appear larger (scale bar shown) than its actual physical dimensions.

FIG. 3 shows separation of Mo-NP from intact Mo-MLV, NP and free Mo-MLV membranes on density gradients. Virus alone (top panel), green fluorescent-nanoparticles alone (middle panel) or mixtures of Mo-MLV membranes extruded with the nanoparticles (lower panel) were applied to 0-27% (w/v) dextran (70 kDa) gradients. After centrifugation for 16 hours at 19° C., 0.1 mL fractions were collected and analyzed for fluorescence (open circles) using a 96-well fluorescence plate reader (left axes, expressed as relative fluorescence units) or virus envelope protein by Western blot. Signals on the Western blots were quantified by densitometry using ImageJ software and expressed as arbitrary densitometry units (solid circles, right axes).

FIGS. 4A-4B show electron microscopy of dextran gradient purified nanoparticles and virus-membrane coated nanoparticles. FIG. 4A shows NPs (top row), Mo-MLV virus (second row), liposomes made from pure brain lipids (third row), membranes made from virus (fourth row), and nanoparticles coated with pure lipids (fifth row) or Mo-MLV membranes (sixth row) as analyzed by electron microscopy. Material was adsorbed to Formvar-coated copper grids (400 mesh) by 10 min of incubation at room temperature and then stained with 2% uranyl acetate for 45 s. Excess liquid was removed and the grid was dried and imaged on a Philips 201 electron microscope at 60 kV (Philips Electron Optics, Eindhoven, The Netherlands). The size bar is shown at the top right and is the same for all the images. Arrowheads indicate projections from NPs that were likely due to lipid coating the nanoparticles. For some particles (<1% of the population) large projections were observed (middle image for virus-membrane coated NP), ithers were joined by a thin film (right image for lipid-coated NP).

In FIG. 4B, the diameters of nanoparticles, intact Mo-MLV and nanoparticles coated with pure lipids or Mo-MLV membranes were measured from microscope images. At least 10 images were used per analysis, and the average±standard deviation is shown. One way ANOVA followed by the Turkey-Kramer post-test showed a significant difference (p<0.05) between diameters of NPs alone compared to lipid or virus membrane coated nanoparticles (indicated by asterisk).

FIGS. 5A-5B show specific binding of Mo-NP to cells expressing the mCAT-1 receptor. In FIG. 5A a stable cell line expressing mCAT-1 was made in HEK 293 cells that normally lack receptor. The parent (293 cells) and the receptor (mCAT-1) expressing cell lines were then infected with a recombinant Mo-MLV encoding β-galactosidase, at a multiplicity of infection of 0.2 so that 1 in 5 cells should become infected if expressing the receptor. The cells were then stained for β-galactosidase activity after 2 days (stain appears black). Both HEK 293 cells or mCAT-1 expressing cells were challenged with green fluorescent Mo-NP for 2.5 hours at 37° C. For purpose of visualizing the cell membranes, cells were stained with red fluorescent cholera toxin for 30 min at 37° C. and then imaged by fluorescence microscopy. In FIG. 5B binding efficiency was determined by counting the number of Mo-NP bound to either HEK 293 cells or mCAT-1 expressing cells. A total of 120 cells were analyzed per cell line. The average number of particles bound per cell±standard deviation are shown.

FIGS. 6A-6B show receptor-dependent endocytic uptake of Mo-NP into cells. Mo-NPs made with blue fluorecent nanoparticles (identical surface composition to green nanoparticles) were incubated with cells expressing red fluorescent protein-tagged mCAT-1 and GFP-tagged caveolin. After 2.5 hours, cells were fixed and images were taken using a Zeiss LSM 510 UV Meta confocal microscope. In FIG. 6A, serial optical sections were made of cells and one set is shown for one representative cell. The spacing between each section is shown at top left of each image. Composite images of blue (NP), green (caveolin) and red (mCAT-1) fluorescence signals are shown. Overlap of blue and red gives purple (some examples indicated by purple arrowheads) and overlap of all three signals gives white (white arrowheads). FIG. 6B shows separate fluorescence images for blue, green and red channels for midsections at 3 and 4 μm below the surface of the cell.

FIGS. 7A-7B show that Mo-MLV-membrane-coated NPs penetrate and deliver a cargo into the cell cytosol. β-Lactamase (βlac) was covalently coupled to nanoparticles by peptide bond formation using EDC. NPs were separated from free βlac enzyme by centrifugation. Specific activity of the βlac coupled nanoparticles was determined by assaying enzyme activity using nitrocefin, a chromogenic substrate that changes color from yellow to orange when cleaved by βlac. In FIG. 7A, a standard of 1 mg/mL solution of βlac (2441 benzylpenicillin U/mL) was titrated in 2-fold serial dilutions (βlac alone) and compared to NPs alone or NPs coated with βlac (Mo-βlac-nanoparticles). Specific activity of the βlac-NP was calculated using the standard curve shown (lower panel), and the regression coefficient (R²) for the fitted curve is given. Absorbance at 600 nm was measured for each enzyme dilution standard after reaction with nitrocefin for 30 min. Activity was 43.6±12.9 benzylpenicillin U ml⁻¹ μL⁻¹ of NP.

In FIG. 7B, βlac-coupled fluorescent nanoparticles were coated with Mo-MLV membranes (Mo-βlac-NP) and purified. The Mo-βlac-NPs were applied to cells expressing red fluorescent protein-tagged mCAT-1 for 3 h. Cells were then loaded with the fluorescent βlac substrate CCF2/AM and imaged after 2 h. Punctate green fluorescence of NPs and the diffuse green fluroscence of uncleaved CCF2 are seen (left panels). Red fluorescence from mCAT-1 and blue fluorescence from βlac cleaved CCF2 are shown at right.

FIG. 8 shows specific interaction of virus env-coated nanoparticles with receptor expressing cells and endocytosis of nanoparticles. Cells expressing a GFP-tagged caveolin or Rab7 were transfected with a red fluorescent protein tagged Fr-MLV receptor (CAT-1). Some cells were not transfected (green only at the right of the first panel). Fr-MLV env-coated nanoparticles (blue fluorescent) were added and incubated 4 h after which the cells were fixed with fresh 1% paraformaldehyde in PBS and visualized using confocal microscope. Mid-sections of cell cytoplasm are shown with the representative nanoparticles present within endocytic vesicles (arrows). Left panel shows co-association of nanoparticle, receptor and caveolin (white color). Central panel shows nanoparticles within receptor positive endosomes (red/blue). Right panel shows a nanoparticle that has entered a late endosome (Rab7 positive green/blue). Clusters of nanoparticles are due to uptake of multiple nanoparticles or convergence of multiple endosomes as the initial preparation was monodisperse and early time points show single nanoparticles bound to cells. The result presented herein showed that the nanoparticles could be detected within endocytic compartments and identified these compartments in addition to the specific and efficient targeting of the receptor expressing cells by these nanoparticles.

FIGS. 9A-9B show tracking of endocytic vesicles in live cells. FIG. 9A shows expression of recombinant GFP-tagged Rab5 protein (labels early endosomes) in cells by retroviral vectors. Vesicle movement was seen in a series of 1 second frames taken from a movie. A representative vesicle is indicated (arrowhead). Motion of this is apparent. The asterisk is a reference point. FIG. 9B shows detection of early endosomes by GFP-Rab5 expression. The NC endocytic pathway was identified by vesicles not associated with caveolin but stained with labeled cholera toxin B-subunit (right). Nuclei were DAPI stained.

FIG. 10 shows the effect of overexpression of dominant negative (DN) Rab5, Rab7 and Eps 15 genes on entry of Vesicular stomatitis virus, Fr-MLV and VEEV. Each DN gene was expressed in cells using a retroviral vector. Entry was examined using a virus entry assay. The DN mutants may be used to study the entry route taken by the env-nanoparticles and should be similar to the envelope donor virus.

FIGS. 11A-11B show results of cytosol penetration assay. Nanoparticles coupled to β-lactamase (β-lac), using an EDC reaction were purified away from free enzyme on dextran gradients. Activity was assayed using nitrocefan (FIG. 11A, red color). Nanoparticles then coated with Fr-MLV envelope (FIG. 11B) were incubated with cells expressing the receptors (red) and stained with CCF2/AM, a fluorescent β-lactamase substrate (turns blue on enzyme action). Left: Cells+nanoparticles with no β-lac; Right: β-lac+.

FIG. 12 is a schematic representation of the vesicle-mediated endocytosis.

FIGS. 13A-13B are schematic representations of the envelope coated nanoparticle described herein. FIG. 13A shows simple specific-targeting nanoparticle and FIG. 13B shows a more complex specific targeting nanoparticle.

FIG. 14 shows carboxylate modified NPs that were coated by extrusion. Size was determined in a Zetatrac particle size analyzer. 5 independent measurements were made for each sample. Intact NPs (peak A) were compared to VSV-env coated NPs (peak B). An average size increase of 40 nm was observed.

FIGS. 15A-15B show Vero cell membranes were stained with Cholera toxin subunit B (red) to see the cell periphery and internal vesicles. Similar numbers of fluorescent green-yellow NPs that were intact (FIG. 15A) or modified by extrusion with VSV membranes (FIG. 15B) were incubated with Vero cells for 30 min. Both were NP types were blocked with BSA before incubation with cells. Cells were then washed 3 times with normal growth medium and images were taken on a Leica DMIRB inverted epifluorecence microscope fitted with a 100× objective. Images were analyzed by using ImageJ software.

FIGS. 16A-16E are time-lapse fluorescent micrographs. Cells were stained with cholera toxin B subunit (green) to see cell membrane and vesicles. VSV env-coated red fluorescent NPs were incubate with Vero cells for 20 min. Images were then acquired using a TE2000 Nikon inverted microscope fitted with a 100× oil immersion objective and a Cool-SNAP HQ cooled CCD camera. Exposure time was 100 ms per wavelength and 20 seconds between each set of exposures. Images sequences were compiled and analyzed using ImageJ software. FIGS. 16A-16D are representative images where each is separated by 3 minutes. FIG. 16E shows trajectories of NPs mapped by ImageJ software using “Particle tracker” plugin.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “contacting” refers to any suitable method of bringing the composition described herein and an anti-viral agent or combination thereof into contact with a virally infected cell. In vitro or ex vivo this is achieved by exposing the infected cell to the composition in a suitable medium. For in vivo applications, any known method of administration is suitable as described herein.

As used herein, the term “nanoparticle” refers to a hollow or solid spherical or irregular particle with sub-micrometer dimensions typically but not limited to between 1 to 300 nm.

Viruses have evolved to become highly efficient cell-targeting and cell-membrane penetrating machines. Each virus seeks out appropriate cells to infect among a myriad of potential targets. Viruses have overcome this problem by acquiring envelope proteins (envs) that play key roles in entry into the cell. Envs specifically bind to a single or a set of cellular receptor molecules, stimulate uptake of the virus and finally, mediate penetration into the cytosol by driving virus-cell membrane fusion. This interaction allows the virus to overcome the barrier of the cell membrane and introduce its genome into the cell cytoplasm where it can replicate. Retrovirus pseudotypes, retrovirus cores with the envelope proteins of different donor virus, have been shown to enter cells identically to the env donor (1-2). These observations indicated that specific env-virus core interactions are unimportant, and so it should be possible to separate the envs away from a native virus particle while keeping the receptor-targeting and entry mechanisms intact.

There have been limited previous attempts to use virus envelopes to target vesicles or nanoparticles. Most work to harness the potential of viral envelope proteins has focused on using Influenza A to make “virosomes,” which are virus-derived vesicles made by detergent extraction of virus and subsequent detergent removal. Just like virus, Influenza A-derived virosomes bind cell membranes through sialic acid modifications on membrane proteins and cause membrane vesicle fusion at acidic pH. Additionally, mixtures of Sendai virus and more recently, recombinant Hemagglutinating virus of Japan-DNA aggregates have also been used to enhance transfection of DNA into cells. Furthermore, virosomes have also been prepared with envelope proteins of vesicular stomatitis virus (VSV), human immunodeficiency virus (HIV), and herpes simplex virus, but in all cases cell entry was not evaluated (3-4). However, the bulk of work has mainly focused on Influenza A, because, in general, the Influenza A envelope protein is an exception and tolerates solubilization in detergents. Unfortunately, most other envelope proteins disintegrate into their subunits upon detergent extraction and lose the ability to fuse cell membranes. Accordingly, methods utilizing these envelope proteins had a very limited applicability and lacked the capacity to convey cargoes to selected cellular and subcellular targets.

Certain viruses can target specific cell types through env interactions with cell type specific receptors or receptor combinations. Moloney murine leukemia virus (Mo-MLV) is one example of the exquisite tissue and cell selectivity that can be achieved by viruses. This retrovirus infects only cells that express the integral membrane protein, mCAT-1, which is found on many mouse cell types. Human cells cannot be infected with Mo-MLV. However, when mCAT-1 is expressed on normally resistant human cells, they become susceptible to infection to give >10⁶ infectious virus units/ml while neighboring cells lacking receptors remain uninfected (5). A related retrovirus to Mo-MLV is Human Immunodeficiency Virus (HIV), a retrovirus that only infects a subset of cells that express CD4 and CXCR4 or CCR5 chemokine receptors. This combination of proteins is commonly found on T cells or monocyte-derived cells, respectively. Cells lacking these receptor combinations are not infected efficiently by HIV (6). Thus, a method to coat nanoparticles with functional envs of these receptor specific viruses would permit targeting of distinct populations within the host, something that Influenza A-derived virosomes do not permit.

The present invention discloses a method to coat nanoparticles with the envs of Mo-MLV and shows that these particles mimicked virus in binding to cells bearing specific receptors. Additionally, these particles did not interact with bystander cells that lacked appropriate receptor. It is also demonstrated herein that the env-derivatized nanoparticles were capable of delivering an enzyme cargo into the cytosol of the cells, possibly through an endocytic route. The method described herein did not use detergents but instead, the envelope protein containing membranes were directly coated onto nanoparticles by extrusion. Extrusion is the process of forcing material through a small rigid orifice. The resulting pressure and mechanical shear force breaks the material into smaller particles. It is commonly used to prepare homogenous populations of unilamellar liposomes out of multilamellar lipid sheets. It was hypothesized herein that extruding nanoparticles together with virus membrane sheets could coat a thin film of membrane over the surface of the NP. Although Mo-MLV envelope proteins were used herein, envelope proteins from other viruses can be used to diversify the targeting potential of the nanoparticles.

Of the many nanoparticles that are available, the present invention used commercially available, highly fluorescent, carboxylate-modified nanospheres. The following are the reasons for using the highly fluorescent, carboxylate-modified nanoparticles in the present invention. First, retrovirus cores are approximately 100 nm in diameter, as is the nanoparticle. This means that they should be able to physically enter the same endocytic pathways as a native virus. Second, retrovirus cores are electron-dense structures and are relatively rigid. Capsid cores are also spherical and have no icosohedral symmetry as seen by electron microscopy (transmission or cryo-em) and therefore a spherical polymer bead is likely a good substitute for the capsid.

Additionally, these types of nanoparticles have similar chemical and physical properties as a retroviral nucleocapsid (virus core), being a partially negatively charged, hydrophobic sphere 100 nm in diameter. The carboxylate modified nanoparticles are a good approximation of this core, having an overall negative charge and partial hydrophobic patches on unmodified surfaces. Since similar nanoparticles can be obtained with different chemical adducts varying in absolute charge and hydrophobicity, the present invention contemplates examining the role of chemical composition of the nanoparticle on targeting. Additionally, generation of novel nanoparticles with specific chemical compositions and membrane coating efficiency that can harbor cargoes including drugs is contemplated.

With regards to immunogenicity of the virus envelope-coated nanoparticles, most of the virus envelopes are poor immunogens unless genetically manipulated. Virus envelopes are therefore well suited for nanoparticle targeting and immune evasion. Most virus envelopes elicit weak or short lived responses and cloak crucial epitopes with sugar modifications. Furthermore, since many virus substrains exist that differ in their spectrum of exposed epitopes, it would be practical to change the envelope subtype between nanoparticle-based treatments without altering target specificity or function but avoiding neutralization by antibodies or cell-based immune responses.

Additionally, pseudotyped virus generated using the method described herein can be safely administered without concerns of infection. The system described herein is essentially the same as that used for retrovirus-based gene therapy, except that the genetic component of the virus is eliminated herein. Retrovirus-based systems have been extensively studied and considered safe enough for human trials. Removal of the genetic component makes them even safer, eliminating the potential for genetic alteration of the targeted cell.

While the envelopes chosen may not be as good immunogens, they may serve to enhance vaccine productivity by delivering nanoparticle antigen cargoes (proteins, peptides or DNA encoding antigens) to antigen presenting cells. Two such targets are dendritic cells and macrophages. These cell types are important for antigen presentation in establishing robust cell-based immune responses. In order to target specific immune cells, one may coat the nanoparticle with the envelope proteins of viruses that demonstrate high tropism for such cells. For instance, the Venezuelan equine encephalitis virus (VEEV) shows a high tropism for dendritic cells such as Langerhans cells in the skin. Therefore, Venezuelan equine encephalitis virus env-coated nanoparticles may be used for delivery of immunogens or immunostimulatory cargoes to such cells.

Another virus that shows macrophage specificity is HIV. The envelope of HIV may be manipulated and used to coat nanoparticles using the same method as described herein. These nanoparticles coated with the envelopes of HIV would be ideal for delivery of cargoes to mucosal macrophages lining the genital tract. One may also use the envelope of Ebola virus to coat nanoparticles and use them in the delivery of cargoes.

Regarding the source of the virus envelope material, pseudotyped particles may be used instead of using a cell based expression system, which may require a further purification step. The pseudotyped particles are a source of envelopes that are far superior to membranes produced using the cell based expression system for the following reasons: First, the envelopes are enriched on the particle's surface, to the exclusion of other extraneous membrane proteins. Second, the pseudotyped particles enter cells and therefore the envelopes on their surfaces must be properly folded and functional. Third, the viral membrane is loosely and non-specifically associated with the underlying viral matrix and is easily separated and recovered.

The present invention demonstrated that preparation of the Mo-MLV particles provided more than sufficient envs to perform >10 independent NP coatings. Since each batch contained tens of thousands of nanoparticles there should not be a problem with supply. It is contemplated that the other virus envs may be obtained in similar amounts from pseudotyped particles. Derivatized nanoparticles should then be readily obtained. These are likely to function just as well as the Mo-MLV particles as the envs share the same basic physical properties. Use of other types of murine leukemia virus having different receptor specificities is contemplated since each of these viruses is closely related and has similar physical properties. These include but are not limited to xenotropic, amphotropic and polytropic viruses and they may behave identically to the ecotropic Mo-MLV. However, the use of cell membranes as a scalable source of envs is also contemplated by coupling it with purification schemes to increase the specific activity and constrain the orientation of the envs on the nanoparticles. The envs will be extracted from the cell membranes with two newly available detergents that do not appear to disrupt env subunit association. The proteins will then be affinity purified directly onto avidin or antibody-coated nanoparticles. This approach will allow the assessment of different sources of envs to modify the NPs and provides the proposal with greater scope and additional avenues to translate the work into a practical application.

The present invention used a lipid-labeling agent (DilC₁₈) to identify particles that were coated with virus membranes. Since the incorporation of this label may be disruptive for virus env-cell interaction, a lipophilic dye was used only when analyzing the composition of the coated NPs. The env-nanoparticle association and purification for Mo-MLV where env-coated nanoparticles are identified as a distinct fraction on the density gradients are optimized when using this dye. This overcomes the need to include the label when making the coated nanoparticles.

Agglomeration of the nanoparticles due to non-specific nanoparticle-nanoparticle interaction is a potential problem. This would preclude the use of such particles in further targeting analysis, as aggregates would likely behave differently to single particles. It was observed that aggregated particles when present have a higher density than single particles and can be effectively separated from single particles on density gradients. The single particles are found in the top third of the nanoparticle+virus env peak on the gradient. These remain as single particles for more than 1 week at 4° C., when 1 mg/ml BSA is added as a stabilizer. The particles behave equivalently to freshly made particles in cell binding studies. As part of the analysis of the nanoparticles, the aggregation state of the nanoparticle is also assessed by microscopy. This differentiates signal emitted by single particles versus that of aggregates.

In summary, nanoparticles have considerable potential for use in biology and medicine, including the delivery of cargoes of antigens, antigen-encoding nucleic acids or therapeutic agents. However, without specific targeting many of these attempts will be unsuccessful. The present invention embodies methods for coating nanoparticles with virus envelopes containing specific proteins that facilitate the targeting to specific cells and cellular entry pathways. The viral envelope coated nanoparticles are shown in FIGS. 13A and 13B. The examples of virus whose envelopes may be used to coat such nanoparticles may include but are not limited to Retroviruses such as Moloney murine leukemia virus (Mo-MLV), Friend murine leukemia virus (Fr-MLV), other types of MLVs and HIV, Togaviruses such as Venezuelan Equine Encephalitis virus (VEEV), Filoviruses such as Ebola virus, Herpes viruses such as Herpes simplex, Varicella Zoster, Cytomegalovirus and Karposi's sarcoma virus, Arenaviruses such as Lassa Fever virus, Pox viruses such as Vaccinia or Smallpox, Coronaviruses such as SARS, Flaviviruses such as West Nile virus, Rhobdoviruses such as Rabies and Vesicular stomatitis virus, Paramyxoviruses such as Measles and Repiratory syncytial virus and Orthomyxoviruses such as Influenza A.

The approach of targeting nanoparticles to the cells, targeting specific entry mechanism and subcellular structures described herein is unique. This approach used herein can be exploited to activate chemicals, with the potential to substantially decrease systemic toxicity. The examples of the cargo that the viral envelope coated nanoparticle of the present invention can carry may include but are not limited a protein of a pathogen, a modified protein of the pathogen, a nucleic acid or a nucleic acid like molecule encoding an immunogenic peptide, an antigen or an inhibitory RNA, a protein, a probe or a therapeutic agent. It is also contemplated that the viral envelope coated nanoparticle of the present invention may be used in diagnostic assays for pathogens without the risks associated with the exposure to competent infectious pathogens.

The present invention is directed to a composition, comprising a biodegradable core particle having a diameter of at least 100 nm, and partial hydrophobic properties on unmodified surface of the core particle and a coating comprising one or more than one viral envelope proteins. This composition may further comprise a protein of a pathogen, a modified protein of the pathogen, a nucleic acid or a nucleic acid like molecule encoding an immunogenic peptide, an antigen or an inhibitory RNA, a protein, a probe or a therapeutic agent. Examples of the therapeutic agent may include but are not limited to a chemotherapeutic agent, a toxin, an immune stimulant, a cytotoxic agent or a radioisotope. The particle may bear a negative or a positive charge or motif to facilitate interaction with the viral envelope protein(s). Additionally, the core particle may be fluorescently labeled. The viral envelope protein may comprise virus specific targeting protein to cellular plasmalemma receptors, virus specific targeting protein to cellular internal structures or a combination thereof. Furthermore, the viral envelope protein may include but are not limited to an envelope protein of Retroviruses such as Moloney murine leukemia virus (Mo-MLV), Friend murine leukemia virus (Fr-MLV) and other types of murine leukemia viruses and HIV, Togaviruses such as Venezuelan Equine Encephalitis virus (VEEV), Filoviruses such as Ebola virus, Herpes viruses such as Herpes simplex, Varicella Zoster, Cytomegalovirus and Karposi's sarcoma virus, Arenaviruses such as Lassa Fever virus, Pox viruses such as Vaccinia or Smallpox, Coronaviruses such as SARS, Flaviviruses such as West Nile virus, Rhobdoviruses such as Rabies and Vesicular stomatitis virus, Paramyxoviruses such as Measles and Repiratory syncytial virus or Orthomyxoviruses such as Influenza A. Examples of the core particle may include but are not limited to hollow or solid, polystyrene particles, latex particles, dextran derivatives, cellulose derivatives, and other organic conjugates.

The present invention is also directed to a method of generating the viral envelope coated core particle discussed supra, comprising: lysing an intact virus via osmotic shock, sonicating membrane of the virus to dissociate viral envelope and nucleocapsid of the virus, separating the viral envelope and the nucleocapsid of the virus using a density gradient, incubating the viral envelope and the core particle for at least fifteen minutes, sonicating the viral envelope/core particle mixture to dissociate envelope vesicle aggregates and to permit association of the envelope with the core particle and passing the virus envelope/core particle mixture through an extruder with a defined pre size of 50 to about 200 nm such that the passage through the filter and pressure applied during the passage forces the membrane of the virus to be extruded over the core particle, thereby generating the viral envelope coated core particle. This method may further comprise attaching a fluorescent label to the viral envelope coated core particle. This method may also further comprise loading the viral envelope coated core particle with a protein of a pathogen, a modified protein of the pathogen, a nucleic acid or a nucleic acid like molecule encoding an immunogenic peptide, an antigen or an inhibitory RNA, a protein, a probe or a therapeutic agent. Examples of the therapeutic agent are the same as discussed supra.

The present invention is further directed to a targeted therapy to an individual, comprising administering the above-discussed composition to the individual, where the viral envelope protein in the composition targets the composition to the specific receptors on a cell, to specific cellular entry mechanisms within the targeted cell or to a combination thereof. The type of cell targeted by such a method may include but is not limited to an immune cell, a cancer cell, a cell infected by a pathogen, dendritic cells and other antigen presenting cells, cells of the liver and spleen, neurons and cells lining blood vessels including the blood-brain barrier.

The present invention is still further directed to an immunogenic composition comprising the above-discussed composition, where the composition comprises a nucleic acid or a nucleic acid-like molecule encoding an immunogenic peptide or an antigen, an immunogenic peptide, a protein or an immune stimulant.

The present invention is also directed to a method of delivering an immunogenic composition to an immune cell in an individual, comprising: administering the above-discussed immunogenic composition to the individual, where the viral envelope protein in the composition binds specifically to the immune cell, thereby delivering the immunogenic composition to the immune cell in the individual. The immune cell may be a dendritic cell or a macrophage.

The present invention is further directed to a kit, comprising: the above discussed composition, where the composition comprises a protein of a pathogen or a modified protein of the pathogen.

The present invention is still further directed to a method of detecting an infection caused by a pathogen in an individual, comprising: obtaining a biological sample from the individual and contacting the biological sample with the kit discussed supra, thereby detecting the infection caused by the pathogen in the individual. Examples of the biological sample may include but is not limited to serum, spinal fluid, saliva and urine and that of the infection detected by such a method may include but is not limited to the infection caused by any envelope viral agent such as West Nile virus, SARS, Venezuelan equine encephalitis virus, HIV, Herpes, Measles or Cytomegalovirus, Influenza or Chicken pox.

The composition described herein and other anti-viral agents can be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of the composition described herein may comprise conventional non-toxic, physiologically or pharmaceutically acceptable carriers or vehicles suitable for the method of administration.

The composition described herein may be administered independently one or more times to achieve, maintain or improve upon a therapeutic effect. It is well within the skill of an artisan to determine dosage or whether a suitable dosage of either or both of the composition and anti-viral agent comprises a single administered dose or multiple administered doses. An appropriate dosage depends on the subject's health, the efficient targeting of the components to the specific cell and/or tissue, the route of administration and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Virus and Cell Lines

The Moloney strain of ecotropic Murine Leukemia Virus (Mo-MLV) was collected from CL-1 cells supplied by Dr. J. Cunningham (Harvard Medical School). These cells continually secrete virus into the culture medium. American Type Tissue Culture Collection (ATCC) provided HEK 293 cells. Clones expressing HA-tagged or red fluorescent (mStrawberry)-tagged mCAT-1 were generated by transfection with expression plasmids. Transfected cells were selected by treatment with G418 and colonies were isolated and characterized. GFP-tagged Caveolin expressing cell lines were generated by transfection of expression plasmids followed by selection in blasticidin. For uptake experiments, the GFP-caveolin expressing cells were transiently transfected with the mStrawberry-tagged mCAT-1 expression plasmid and assays were performed 48 hrs later. Expression vectors were pcDNA3 and pLENTI (both from Invitrogen, CA) for mCAT-1 and caveolin, respectively. All cell lines were grown in Dulbecco Modified Eagle Medium (DMEM) from Invitrogen and supplemented with 10% Fetal Bovine Serum (Gemini Bioproducts, CA), penicillin (200 U/ml), and streptomycin (200 mg/ml) at 37° C. and 5% CO₂.

### Example 2

### Nanoparticles

Fluorescently labeled 100 nm diameter nanospheres were purchased from Invitrogen. Both green fluorescent (yellow-green, excitation 505 nm and emission at 515 nm, #F8803) and blue fluorescent (350 nm excitation and 440 nm emission, #F8797) carboxylate modified nanospheres (2% solids) were used.

### Example 3

### Plasmid Constructs

The caveolin construct was provided by Dr. Lisa Elferink (University of Texas Medical Branch), and the plasmid encoding the mStrawberry protein was provided by Dr. R. Tsien (University of California at Los Angeles). mStrawberry was cloned into an expression plasmid (pcDNA3) to give an in-frame c-terminal fusion with mCAT-1. For this, the original C-terminal HA-tag was excised with XhoI and ApaI, and was replaced with mStrawberry digested with XhoI and PspOMI restriction endonucleases. The primers used to PCR amplify the mStrawberry gene from the original vector were 5′: GATCTCGAGCGTGAGCAAGGGCGAGGAGAATAACATGG (SEQ ID NO: 1) and 3′: TCAGCGGCCGCTACTTGTACAGCTCGTCCATGCCGCCG (SEQ ID NO: 2). The XhoI endonuclease site used for attachment to mCAT-1 is underlined.

### Example 4

### Virus Membrane Preparation and Extrusion onto Nanoparticles

Mo-MLV were lysed in a hypotonic buffer consisting of 1 mM EDTA and 10 mM HEPES, pH 7.4, followed by sonication on ice. A probe sonicator (Misonix, NY, model: XL ultrasonic processor with a CL4 probe) was used with five pulses of ten seconds each at 30% power. Sucrose was added to 0.25M, and intact virus and the cores were pelleted by centrifugation at 20,000×g for 1 hr at 16° C. The virus membranes remaining in the supernatant were pelleted by centrifugation at 100,000×g for 2 hours at 4° C., and the pellet was resuspended in Dulbecco's Phosphate-Buffered Saline (PBS) from Cellgro, MO. A 100 μl aliquot of virus membrane suspension was incubated with 1 μl NP stock (F8803 or F8797 from Invitrogen, CA) and diluted up to 1 ml with PBS for 15 minutes. The resultant solution was sonicated four times in 30 second pulses with a Branson E-Module Ultrasonicator at full power. Immediately following sonication, the mixture was passed 40 times through an Avanti mini-extruder (Avanti Polar Lipids, Inc., CA) equipped with a Whatman 0.2 mm polycarbonate membrane (Fisher Scientific) flanked on each side by a filter support (Avanti Polar Lipids, Inc., CA). After extrusion, Bovine serum albumin (Sigma-Aldrich, MO) at 1 mg/ml was added to decrease non-specific interactions.

### Example 5

### Density Gradients

Dextran (70 kDa) from Leuconostoc mesenteroides (Sigma-Aldrich, MO) was added to PBS to make density gradients from 2%-27% (w/v) with the top 0.5 ml being overlaid with the extruded virus/nanoparticle mixture. The gradients were centrifuged at 70,000×g for 16 hours at 19° C. in a Beckman SW55Ti rotor. Fractions (0.1 ml) were taken from the top and dispensed into a 96 well plate for fluorescence analysis by a Molecular Devices SPECTRAmax M2 plate reader.

### Example 6

### Beta-Lactamase Coupling

The 0.1 μm (505/515) fluorescent carboxylate-modified nanospheres (Invitrogen, CA) were coupled to β-lactamase through peptide bond formation using 1-Ethyl-3-(3-dimethylaminopropyl)-carbodiimide (Pierce, Ill.) reaction suggested by Molecular Probes. In short, 10 μl NP were diluted into 100 μl of 50 mM MES, pH 6.5 with 1 mg/ml penicillinase from B. cereus (cat# PO389; Sigma-Aldrich, MO) and incubated for 15 min. EDC was added to 4 mg/ml and allowed to react for 2 h, followed by quenching with 0.3 M glycine, pH 7.4 (Sigma-Aldrich, MO). Nanoparticles were isolated by pelleting at 25,000×g for one hour at 4° C. in an Eppendorf 5417C Centrifuge. Three washes of PBS were performed with pelleting as described above between each wash. After the final wash, the modified nanoparticles were resuspended in 100 μl of PBS supplemented with 0.1% (w/v) sodium azide.

### Example 7

### Fluoescence Microscopy

Cells were fixed in 2% paraformaldehyde, pH 7.4 at 22° C. for 30 minutes. Initial imaging of NP binding to cells and cytosolic β-lactamase activity was performed with a LEICA DMIRB inverted microscope. Confocal microscopy was performed using a Zeiss LSM 510 UV Meta laser scanning confocal microscope.

### Example 8

### Visualization of Cytosolic β-Lactamase Activity

The Invitrogen GeneBlazer Detection kit was used for visualization of cytosolic β-lactamase as an indication of NP penetration into the cell cytosol. Briefly, cells were incubated with Mo-βlac-NP for 3 hours, followed by a rapid wash with PBS. The cells were then loaded with CCF2/AM supplemented with 1 mM probenecid for two hours at room temperature, and were monitored on an LEICA DMIRB inverted epifluorescence microscope.

### Example 9

### Antibodies

Antibodies specific for the envelope protein of Mo-MLV (ATCC Number VR-245) and a secondary goat-anti-mouse-HRP antibody (Pierce, Ill.) were used for detection of virus envelope proteins on Western blots.

### Example 10

### Statistics

Statistic analysis was performed using Graphpad software (GraphPad Prism version 4.00 for Windows, GraphPad Software, San Diego, Calif., www.graphpad.com). Data were compared by one way ANOVA and included the Turkey-Kramer post test.

### Example 11

### Results

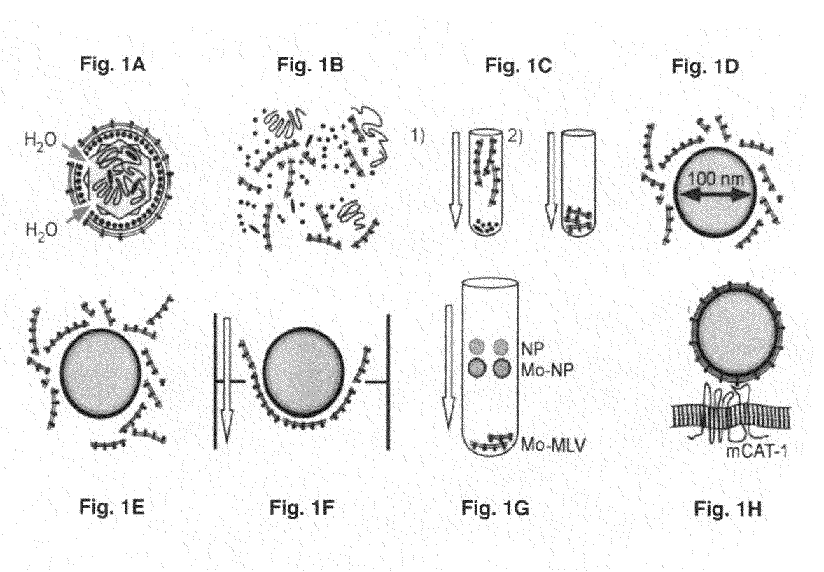

The method to prepare virus membrane-coated NPS from virus is shown schematically in FIG. 1. Purified Mo-MLV were first osmotically shocked and then membranes were released by sonication. Intact virus and virus copres were then pelleted away from the membranes and soluble proteins by centrifucation at 20,000 g in sucrose. The membranes remaining in the supernatant were collected by pelleting them by centrifugation at 100,000×g. This viral membrane preparation was incubated with nanoparticle, sonicated to dissociate env vesicle aggregates and then passed 40 times through a miniextruder equipped with a 0.2 μm membrane. Experiments with fluorescently-labeled pure lipid membranes indicated that 94% of the NPs were coated (FIGS. 2A-2B). Addition of 1 mg/ml BSA at the completion of extrusion prevented non-specific interactions of NPs with cells.

To separate Mo env-coated nanoparticles (Mo-NPs) from uncoated nanoparticles and free virus membranes, the extruded mixture was applied to a 0-27% dextran (70 kDa) gradient (FIG. 3). Dextran is advantageous as it does not contribute significantly to buffer osmolarity, but forms robust density gradients. The sedimentation of green fluorescent nanoparticles in the gradient was detected in fractions with a fluorescence plate reader, and the envelope proteins were detected by Western blot analysis using antibodies specific against Mo-MLV envelope protein. Virus allone pelleted to the base of the gradient (FIG. 3, top). Uncoated nanoparticles migrated at a lower density fraction in the middle of the gradient (FIG. 3, middle). In contrast, virus membranes extruded with NPs gave a single peak of fluorescence that was intermediate between the Mo-MLV pellet and the untreated NP peak fractions (FIG. 3, bottom). Virus envs were detected in low-density fractions corresponding to frr env protein and virus membranes. Envs were also present in the fraction corresponding to peak NP fluorescence. This shift of the NP peak and its comigration with virus envs indicated that the density of the NPs was altered and suggested that NPs were associated or coated with virus membranes. The extent of the shift also indicated that the coating was as efficient as for pure lipids and gave means to separate the products of coating from the starting materials.

Electron microscopy was performed to further characterize the products of extrusion and gradient purification. Images of NPs, virus, lipid membranes, virus membranes, and extruded material were analyzed. NPs had an average diameter of 100±6 nm (FIG. 4A, first row). Mo-MLVs were less regular with an average diameter of 144±9 nm, which is typical for this virus. For some virus particles, images of sufficient clarity to observe env proteins as small spikes projecting from the surface of the virus particle (FIG. 4A, second row). The nucleocapsids of the Mo-MLV were more uniform and had similar dimensions as the NPs. The pure lipids were visible as irregular unilamellar and multilamellar sheets and vesicles that ranged in size from 100 to 500 nm acoress (FIG. 4A, third row). This is consistent with the spontaneous formation of liposomes that occurs after lipids are hydrated. The purified virus membranes adopted shapes similar to those seen with the purified lipids but formed smaller structures of typically 50-200 nm (fourth row). For NPs extruded with lipids or virus membranes, mos NPS appeared to be at least partially bounded by a thin membrane. A subset of NPs was apparently held together by a connecting membrane (FIG. 4A, fifth row, last image). Most NPs had obvious projections or bumps, suggesting that lipids were more loosely associated at these points or other material was trapped under the surface. For some NPs (<1% of the population) coated with virus membranes, larger proportions were also visible and appeared to be comprised of a loosely associated virus membrane. The increase in average diameter of the NPs after pure lipid or virus membrane coating was also apparent with average diameters of 107±2 and 109±6 nm, respectively (FIG. 4A, last two rows). This small size increase (average increase of 7-9 nm) was statistically significant (p<0.05). Given that a hydrated lipid bilayer has a width of 3.7 to 4.6 nm, the observed increase in diameter likely corresponded to a closely associated lipid bilayer bounding the NP (FIG. 4B). This, it was concluded that the extrusion method was effective in coating the NPs with membranes made from pure lipid as well as virus.

Next, the functionality of the virus-membrane-coated NPs (Mo-NPs) was then examined. Initially, binding experiments were performed to establish that Mo-nanoparticles (Mo-NPs) bound to mCAT-1-expressing cells and not to cells lacking the receptor. Human-derived 293 HEK cells normally lack receptor and completely resist virus infections. When they were transfected with an expression plasmid encoding the mCAT-1 protein, they became highly susceptible to infection (FIG. 5A, top panel). The normal 293 HEK and mCAT-1-expressing cells were then incubated with green-fluorescent Mo-nanoparticles for 2.5 hours (FIG. 5A, bottom panels). For visualization purposes, cell membranes were stained with red fluorescently labeled cholera toxin which binds to the surface and internal cell membranes (7). The number of Mo-NPs bound to either HEK 293 cells or mCAT expressing cells were counted (FIG. 5B). The HEK 293 cells expressing mCAT-1 bound 26-fold more Mo-NP than did cells lacking the receptor. This significant increase (p<0.01) in binding demonstrated that like Mo-MLV virus, the NP had gained high receptor specificity due to coating with the virus membranes. It is expected that this evaluation is an underestimate since clausters of Mo-NPs that were not present prior to addition to cells were counted as one. This cluster may represent trafficking of particles over the surface and internalization into the cell and will be investigated in future.

The ability of envelope protein-derived nanoparticles to enter cells after binding was then assessed by tracking receptor-NP association into cellular endosomes. Endosomes are vesicles that sample extracellular fluid and internalize ligand-bound receptors off the surface as invaginations of cell membranes. Two pathways have been well characterized, and are distinguished in use of clathrin or caveolin protein for vesicle formation. Clathrin- and caveolin-dependent endosomes may then both converge and use similar proteins, e.g. Rab5, for transition to early endosomes (8-11). Caveolin was previously revealed to play a significant role in infection by amphotropic MLV, which differs from Mo-MLV in receptor specificity (12).

To follow association with the receptor and the movement of nanoparticles into cells, an expression plasmid encoding a red fluorescent protein (mStrawberry) tagged mCAT-1 receptor was transfected into cells along with plasmid encoding GFP-tagged caveolin. The cells were then challenged with blue fluorescent Mo-nanoparticles that have identical chemical properties to the green ones. Serial optical images from the top to the base of the cells were then made using confocal microscopy.

As observed earlier, the Mo-NP specifically bound to cells expressing red fluorescent mCAT-1, indicating once again that entry was based on the interaction between Mo and mCAT-1. At the time when cells were fixed, three-quarters of the Mo-NP were present at the cell surface, while the remainer internalized. The optical sections revealed that the NPs below the cell surface had penetrated 3-4 μm into the cell and were most often still associated with receptor (purple arrowheads, FIG. 6A). Some Nps were also associated with caveolin as well as the receptor (white arrowheads, FIG. 6A). This observation indicated that caveolin and therefor caveolae were likely involved in the internalization of some Mo-NP but does not rule out other uptake pathways. More importantly, the Mo-NPs were being taken into cells through a receptor-dependent mechanism, behaving similarly to a virus. Separate fluorescence images were acquired for blue, green and red channels for midsections at 3 and 4 μm below the surface of the cell (FIG. 6B).

Endocytic pathways, such as caveolin-mediated endocytosis, converge at early endosomes where Rab5 plays an integral role in trafficking of cargoes. Consistent with this, wild type Rab5-GFP colocalized with mCAT-SFP and Mo-NP, which were seen both at the membrane of the cell and inside the cytosol. This role of Rab5 in endocytosis of Mo-nanoparticles was supported by the impact of a mutant DN form of Rab5, Rab5-S34N-GFP, which blocks early endosome formation and kept most of the MO-NP at or close to the cell surface together with mCAT-SFP and Rab5-S34N-GFP. The magnification of both sets of images was the same, although there was a rather large cell in relative to the average cell size in both sets of images.

While nanoparticles specifically bound to receptor and were efficiently endocytosed, it remained unclear if any escaped the endocytic compartment, containing receptors, to penetrate into the cellular cytosol, as would be expected if the virus env proteins had remained fully functional. This is a critical feature of any NP-delivery vehicle, for without cytosol access, any application of the coated nanoparticles would be severely limited. To demonstrate that nanoparticles entered the cytosol, fluorescent green nanoparticles were modified with beta-lactamase (βlac) before env coating. The enzyme was covalently coupled to nanoparticles by peptide bond formation using an 1-Ethyl-3-(3-dimethylaminopropyl)-carbodiimide (EDC) reaction according to the NP manufacturer's protocol (Invitrogen, CA) (13). In short, 10 ml NP stock (2% solids) and 1 mg/ml βlac were incubated for 15 min in 50 mM MES, pH 6.5. Peptide bond formation was catalyzed by addition of 4 mg/ml EDC and allowed to react for two hours. The reaction was quenched with 0.25 M Glycine, washed in PBS three times, and resuspended in 0.1% Sodium Azide in PBS for storage at 4° C. The activity of βlac-NP was assessed using the chromogenic substrate nitrocefin, which underwent a color change from yellow to orange when acted on by βlac (14). The activity of βlac-NP was compared to unmodified NP and 2-fold serial dilutions of a 1 mg/ml stock of βlac, and it was determined that βlac-nanoparticles had an enzymatic activity of 43.6±/−12.9 benzylpenicillin units/ml⁻¹ (μL⁻¹ of βlac-NPs) using nitrocefin, achromogenic substrate of βlac (FIG. 7A).

When βlac is ectopically expressed in the cell cytoplasm, activity can be sensitively detected using, CCF2/AM. Initially, CCF2/AM is colorless and non-fluorescent, but after being passively loaded into cells, it is acted on by cytosolic esterases to form CCF2, a highly green fluorescent, water soluble cleavage product of CCF2/AM that is impermeable to membranes. Due to an efficient fluorescence resonance energy transfer (FRET) between two fluorophores, CCF2 emits at 520 nm (green) when excited at 409 nm. However, when βlac is introduced into the cell cytoplasm and cleaves CCF2, the FRET is disrupted and emission drops to 447 nm (blue) (15). This assay has been used to detect entry of HIV into cells by expressing βlac in virus cores (16). CCF2/AM thus, provides a sensitive means to detect penetration of the βlac-conjugated NPs into the cell cytoplasm, which indicates that the envs coating the NPs must have mediated membrane fusion.

The βlac-nanoparticles produced from the EDC reaction were subjected to the same Mo env-membrane coating procedure described above to make green fluorescent Mo-βlac-nanoparticles. The Mo-βlac-NPs or Mo-NPs were then overlaid onto 293 cells expressing red fluorescent protein tagged mCAT-1 and incubated for 3 h at 37° C. Then cells were loaded with CCF2/AM. Loading involved removing the medium containing unbound Mo-blac-nanoparticles and incubating the cells for two hours in a CCF2/AM solution supplemented with the anion transport inhibitor probenecid. Probenecid retained the cleaved CCF2 within the cytosol, which allowed for sensitive detection of blac activity (17). After 1.5 hours, cells were analyzed by confocal microscopy. Only cells treated with Mo-βlac-nanoparticles had blue fluorescence, which indicated cytosolic blac activity (FIG. 7B, right lower panel). Cells incubated with Mo-nanoparticles lacking βlac did not stain blue (right top panels). Residual uncleaved CCF2 present in cells was evident by diffuse green fluorescence, and served as a substrate loading control (left panels of FIG. 7B). The Mo-NP lacking enzyme (FIG. 7B, upper panel) showed a few spots of blue fluorescence, which was identified as bleed-through of optical fluorescence emitted by the clusters of nanoparticles. In other work, saporin, a potent cytotoxin, was coupled to nanoparticles and specifically killed mCAT-1 expressing cells, which indicated that cargo was not restricted to blac. Together, these observations indicated that the nanoparticles delivered cargoes into the cell cytosol. This work demonstrated that the viral env proteins retained membrane fusion capability.

The fusion event that allowed βlac to enter the cytosol was dependent on the receptor-envelope protein interaction followed by triggering of the membrane fusion mechanism of the virus envs. Thus, envelope proteins from other viruses could be used to diversify the targeting potential of the nanoparticles. This is distinct from work with Influenza A virosomes discussed above that bound to cells through surface proteins or molecules modified with sialic acid (oligosaccharide), a ubiquitous protein modification for cells that allows the virus to infect a wide variety of cell types. Likewise, virosomes developed with HSV envelope proteins also infect broad ranges of cells presumably through a multifunctional envelope protein-mediated entry mechanism that is not fully characterized (18-19). HIV virosomes should have greater specificity, but these have been used mainly to induce immune responses and have never been characterized for cell entry (20-21). In contrast, Mo-MLV only enters cells that bear the mCAT-1 receptor, which is not found in the liver (22). Due to this high level of specificity, MLV vectors have been proposed as gene therapy vectors. Additionally, protocols have been developed that allow targeting of cells by making cells express different receptors or by modifying the virus env to contain hormone receptor binding peptides (23-24). Similar methods could be used to target these Mo-nanoparticles to specific cells in human patients.

Furthermore, viruses use cellular endosomes to penetrate into the cells and react to the endosomal environment to trigger release into the cytosol by membrane fusion or disruption. If the endosomal route used by the virus is characterized, then a virus envs could be chosen for delivery of the nanoparticles to specific compartments or regions within the cell. Viruses that rely on pH-dependent entry mechanisms require acidification of endosomes, and must reach very specific pH thresholds before membrane fusion is triggered to release their genomes into the cytoplasm (25-26). Since pH varies depending on the maturation state of the endosome, viruses have found a way to determine precisely the exit point into the cytoplasm. The literature suggests that Mo-MLV enters through a pH-independent pathway and may sense other environmental factors than pH. Mo-nanoparticles and those derived from other pH-independent viruses are then likely to permit access to new endocytic compartments and different regions of the cytosol that cannot be achieved by pH-dependent virus envelope proteins alone. Additionally, many pseudotypes of MLV exist, i.e., viruses that bear foreign envelope proteins on their surfaces, and it should be possible to make nanoparticles out of these, providing a wealth of receptor/cell specificities and biological properties. The virus-membrane coated NPs also provide a new and valuable tool to study and define the entry pathways used by viruses. This will provide key information for the development of new antiviral therapies.

When introduced into an animal, virus-membrane coated NP could have the advantage of avoiding innate or adaptive immune responses that would otherwise remove them from circulation. Virus envs tend to be weak immunogens. This is exemplified in the considerable effort that has been made in making vaccines from virus envelope proteins. Most do not elicit strong immune responses unless genetically manipulated. This lack of immunogenicity is due to carbohydrate modification that can hide crucial epitopes (27). Since many virus substrains exist that differ in their spectrum of exposed epitopes, it would also be practical to change the env subtype between treatments without altering target specificity or function, but avoiding neutralization by antibodies or cell-based immune responses.

Since the method described herein to make the virus membrane-coated nanoparticles is likely not specific to a particular type of NP, virus membranes could be used to encapsulate one of several different nanoparticles that have been tested in vivo, which have promise as therapeutic agents but lack cell specificity. Recently, capsid proteins from Brome mosaic virus were used to encapsulate gold nanoparticles (28). In other work, spherical and rod-shaped DNA cores developed from polyethylene glycol delivered DNA to the cellular cytosol of lung cells (29). A biodegradable core derived of diethylaminopropylamine polyvinyl alcohol-grafted-poly(lactic-co-glycolic acid) (DEAPA-PVAL-g-PLGA) has been shown to decrease the in vivo inflammatory response in the lungs of mice against nano-sized structures (30). To activate an immune response, passive adsorption of recombinantly purified p24 antigen of HIV to poly(D,L-lactide) (PLA) nanoparticles was used to induce high antibody titers against HIV in vivo (31). In each case specific targeting would help to increase treatment specificity and decrease side effects. The use of alternate cores, pseudotypes, and native viruses enhance the method's efficacy, which already serves as a promising base with which nanoparticle cores can specifically target and penetrate cells.

### Example 13

### Nanoparticles Coated with Envelope of Friends Murine Leukemia Virus (Fr-MLV)

Fr-MLV env-coated nanoparticles were generated using the method disclosed supra. The nanoparticles were incubated with cells lacking or bearing a novel red-fluorescent protein-tagged receptor (FIG. 8). Only when cells expressed receptor did the nanoparticle interaction take place. 3D reconstructions of deconvolved stacks demonstrated a fraction of the NPs had penetrated into the cell. The trafficking and penetration properties of such particles were then examined.

### Identification of Subcellular Compartments

The subcellular compartment targeting of nanoparticles by co-localization of particles with specific endocytic markers was examined. FIG. 12 is a summary of major endocytic pathways that will be examined. The endosomes in fixed and live cells were identified using specific staining patterns for the markers listed (FIGS. 9A-9B).

### Use of DN Gene Expression to Dissect Endocytic Entry Pathways

Another method used to dissect the pathways involved in endocytosis of the nanoparticles involved dominant negative (DN) mutant gene expression. Typically, these are GTPases locked in a permanently phosphorylated or dephosphorylated state due to a point mutation in the enzyme active site. When expressed in cells, each blocked the targeted pathway. The function of each DN gene was validated by marker staining patterns (FIGS. 9A-9B) and virus entry assays (FIG. 10).

### Cytoplasm Penetration

The potential of env-coated nanoparticles to penetrate into the cell cytoplasm was also examined. This is a unique feature of virus envs as ligands and is not readily achieved in other systems. However, it is a necessary and key feature of any nanoparticle-based delivery vehicle. To measure entry, nanoparticles were coated with b-lactamase enzyme using an EDC-mediated coupling reaction. Treatment did not change the env coating properties of the nanoparticles. After confirming enzyme activity by a colormetric assay (FIG. 11A), nanoparticles were coated with Fr-MLV envs and added to cells. Penetration was assayed using CCF2/AM substrate which is colorless until metabolized within cell cytosplasm and turns fluorescent green. Action of b-lactamase cleaves the green compound to a blue fluorescent compound (disrupts an internal FRET). With this assay cells became blue only if incubated with nanoparticles containing lactamase (FIG. 11B). This will enable assessment of env-coated nanoparticles for penetration into the cell cytosol.

### Example 13

### Alternative Virus Testing in the Nanoparticle Coating System

Vesicular stomatitis virus (a virus of veterinary concern) has glycoproteins (GP) that promote penetration of virus into a wide variety of cells. This has a membrane that is loosely associated with its core and can be grown to high particle concentrations. Its glycoproteins robust and can be dissolved in detergent and reconstituted into membranes without affecting its function. VSV was purified giving 10× yields (total protein) to that seen with MLV. Membranes were extracted and purified with similar properties and they have been applied onto the carboxylated nanoparticles. When examined in a particle size analyzer, an appropriate size increase was observed, with the peak size shifting from 100 to 140 nm. This would be expected for a single membrane film with protruding VSV GP molecules (FIG. 14).

### Binding of VSV GP-Modified Particles to Cells

The VSV GP-modified NPs were added to cells and compared to particles that had been soaked in BSA only (FIGS. 15A-15B). Time-lapse microscopy was used to track association of the particles with cells over time. Unlike unmodified particles, the NPs attached to the cell surface rapidly. Efficiency matched or exceeded those modified with MLV proteins. Interestingly, the VSV-NPs were seen to track along the cell surface. Many were found associated with filopodia, protruding from the cell surface. Those on the filopodia appeared to move along the filopodia, toward to cell body. This mimics the recently recognized behavior of virus particles to move along filopodia and then enter cells where the filopodia meets the cell body. This data supports the use of these NPs as a virus surrogate. (FIGS. 16A-16E).

### Endosomal Penetration of Modified NPs

Penetration of some NPs into vesicles stained with cholera toxin B subunit was observed. These vesicles may be clathrin-dependent or caveolin-dependent compartments as both are known to take up this marker.

CCF2AM could be used as a marker of endosomal penetration of NPs into the cell cytoplasm. Since one practical use of this technology is to deliver nucleic acids to cells (siRNA or expression plasmids) fluorescently labeled nucleic acids such as quenched fluorescent RNA or DNA oligonucleotides can be used to give a measure of cytoplasm penetration. Both RNAses or DNAses can be found in to the cell cytoplasm with RNAses being more active. Each is commercially available as the basis of RNAse or DNAse detection kits and have optimized sequences. Upon entry into cells, they should become cleaved and generate a strong fluorescent signal that can be measured in a fluorimeter or by microscopy. If so, then this assay can be used instead of the CCF2AM assay.

The present invention discloses the usefulness of virus envelope coats to enhance penetration of nanoparticle cargoes into the cytoplasm of target cells. These modifications will enable efficient delivery of drugs and other cargoes, such as siRNA or plasmids out of the degradative lysosomal pathway and into the cell cytoplasm. The modifications may also enhance particle circulation.

The following references were cited herein:
1. Balliet and Bates, J Virol 1998, 72(1):671-6.
2. Kolokoltsov, et al., J Virol 2005, 79(2):756-63.
3. Daemen, et al., Adv Drug Deliv Rev 2005, 57(3):451-63.
4. Stegmann, et al., Embo J 1987, 6(9):2651-9.
5. Aagaard, et al., J Gen Virol 2002, 83(Pt 2):439-42.
6. Singer, et al., J Virol 2001, 75(8):3779-90.
7. Roepstorff, et al., J Biol Chem 2002, 277(21):18954-60.
8. Haas, et al., Nat Cell Biol 2005, 7(9):887-93.
9. Pelkmans et al., Cell 2004, 118(6):767-80.
10. Pelkmans and Zerial, Nature 2005, 436(7047):128-33.
11. Mundy, et al., J Cell Sci 2002, 115(Pt 22):4327-39.
12. Beer, et al., J Virol 2005, 79(16):10776-87.
13. Shen, et al., Vaccine 2000, 19(7-8):850-61.
14. Jones, et al., Antimicrob Agents Chemother 1981, 20(6):760-8.
15. Campbell, R. E. Trends Biotechnol 2004, 22(5):208-11.
16. Cavrois, et al., Nat Biotechnol 2002, 20(11):1151-4.
17. Zlokarnik, et al., Science 1998, 279(5347):84-8.
18. Johnson et al., J Virol 1984, 52(1):238-47.
19. Spear, et al., Virology 2006, 344(1):17-24.
20. Singh and Bisen, Vaccine 2006.
21. Clapham and McKnight, A. Br Med Bull 2001, 58:43-59.
22. Chang, et al., RNAbiology 2004, 1(2):106-113.
23. Kasahara et al., Science 1994, 266(5189):1373-6.
24. Barnett and Cunningham, J Virol 2001, 75(19):9096-105.
25. Lavillette, et al., J Biol Chem 2006, 281(7):3909-17.
26. Gething, et al., J Cell Biol 1986, 102(1):11-23.
27. Dacheux, et al., J Virol 2004, 78(22):12625-37.
28. Chen, et al., Nano Lett 2006, 6(4):611-615.
29. Fink, et al., Gene Ther 2006.
30. Dailey, et al., Toxicol Appl Pharmacol 2006.
31. Ataman-Onal, et al., J Control Release 2006.

Any patents or publications mentioned in this specification are indicative of the levels of those skilled in the art to which the invention pertains. Further, these patents and publications are incorporated by reference herein to the same extent as if each individual publication was specifically and individually incorporated by reference.

What is claimed is:

1. A composition, comprising:
a biodegradable core particle having a diameter of at least 100 nm—and partial hydrophobic properties on unmodified surface of the core particle; and
a coating comprising one or more than one viral envelope proteins.

2. The composition of claim 1, wherein said composition further comprises:
a protein of a pathogen, a modified protein of the pathogen, a nucleic acid or a nucleic acid-like molecule encoding an immunogenic peptide, an antigen or an inhibitory RNA, a protein, an enzyme, a probe or a therapeutic agent.

3. The composition of claim 2, wherein the therapeutic agent is a chemotherapeutic agent, a toxin, an immune stimulant, a cytotoxic agent or a radioisotope.

4. The composition of claim 1, wherein said core particle has a negative or a positive charge or motif that interacts with the viral envelope protein(s).

5. The composition of claim 1, wherein said core particle is fluorescently labeled.

6. The composition of claim 1, wherein the viral envelope protein comprises virus specific targeting protein to cellular plasmalemma receptors, virus specific targeting protein to cellular internal structures or a combination thereof.

7. The composition of claim 6, wherein the viral envelope protein is an envelope protein of Retroviruses, Togaviruses, Filoviruses, Herpesviruses, Arenaviruses, Pox viruses, Coronaviruses, Rhobdoviruses, Paramyxoviruses or Orthomyxoviruses.

8. The composition of claim 1, wherein the core particle comprises hollow or solid polystyrene particles, latex particles, dextran derivatives, cellulose derivatives, or other organic conjugates and chemical adducts thereof.

9. A method of generating the viral envelope coated core particle of claim 1, comprising:
lysing an intact virus via osmotic shock;
sonicating membrane of the virus to dissociate viral envelope and nucleocapsid of the virus;
separating the viral envelope and the nucleocapsid of the virus using a density gradient;
incubating the viral envelope and the core particle for at least fifteen minutes;
sonicating the viral envelope/core particle mixture to dissociate envelope vesicle aggregates and to permit association of the envelope with the core particle; and
passing the virus envelope/core particle mixture through an extruder with a defined pore size from 50 to about 200 nm such that said passage through the filter and pressure applied during the passage forces the membrane of the virus to be extruded over the core particle, thereby generating the viral envelope coated core particle.

10. The method of claim 9, further comprising:
attaching a fluorescent label to the viral envelope coated core particle.

11. The method of claim 9, further comprising:
loading said viral envelope coated core particle with a protein of a pathogen, a modified protein of the pathogen, a nucleic acid or a nucleic acid-like molecule encoding an immunogenic peptide, an antigen or an inhibitory RNA, an immunogenic peptide, a protein, an enzyme, a probe or a therapeutic agent.

12. The method of claim 11, wherein the therapeutic agent is a chemotherapeutic agent, a toxin, an immune stimulant, a cytotoxic agent or a radioisotope.

13. A method of targeted therapy to an individual, comprising:
administering the composition of claim 1 to the individual, wherein the viral envelope protein in said composition targets the composition to specific receptors on a cell, to specific cellular entry mechanisms within the targeted cell or to combination thereof.

14. The method of claim 13, wherein said cell is an immune cell, a cancer cell, a cell infected by a pathogen, a dendritic cell and other antigen presenting cells, cells of the liver and spleen, neurons or cells lining blood vessels including the blood-brain barrier.

15. An immunogenic composition, comprising:
the composition of claim 1, wherein said composition comprises nucleic acid or nucleic acidlike molecule encoding an immunogenic peptide or an antigen, an immunogenic peptide, a protein or an immunestimulant.

16. A method of delivering an immunogenic composition to an immune cell in an individual, comprising:
administering the composition of claim 15 to the individual, wherein the viral envelope protein in the composition binds specifically to the immune cell, thereby delivering the immunogenic composition to the immune cell in the individual.

17. The method of claim 16, wherein the immune cell is a dendritic cell or a macrophage.

18. A kit, comprising:
the composition of claim 1, wherein said composition comprises a protein of a pathogen or a modified protein of the pathogen.

19. A method of detecting an infection caused by a pathogen in an individual, comprising:
obtaining a biological sample from the individual; and
contacting said biological sample with the kit of claim 18, thereby detecting the infection caused by the pathogen in the individual.

20. The method of claim 19, wherein said biological sample is serum, spinal fluid, saliva or urine.

21. The method of claim 19, wherein the infection is caused by any envelope viral agent such as West Nile virus, SARS, Venezuelan equine encephalitis virus, HIV, Herpes, Measles, Cytomegalovirus, Influenza or Chicken pox.
